(19) United States
(12) Patent Application Publication
(10) Pub. No.: US 2004/0210167 A1
(43) Pub. Date: Oct. 21, 2004

(54) Medical devices containing at least one water-soluble component

(76) Inventor: Sean W. Webster, Port Murray, NJ (US)

Correspondence Address: JAMES D. WITHERS, WITHERS & KEYS, LLC, P.O. BOX 2049, MCDONOUGH, GA, 30253, US

(21) Appl. No.: 10/420,030

(22) Filed: Apr. 17, 2003

(51) Int. Cl. A61B 17/00

(52) U.S. Cl. 601/1; 606/201

(57) ABSTRACT

Medical devices, such as compression sleeves, containing at least one water-soluble component are disclosed. Methods of making, using and disposing of medical devices containing at least one water-soluble component are also disclosed.

### FIELD OF THE INVENTION

The present invention relates generally to medical devices containing at least one water-soluble component.

### BACKGROUND OF THE INVENTION

Deep vein thrombosis (DVT) refers to the formation of a thrombus (i.e., a blood clot) within a deep vein, commonly in the thigh or calf. The blood clot can either partially or completely block the flow of blood in the vein.

DVT occurs when the flow of blood is restricted in a vein. It can be caused by poor circulation because of problems such as heart disease, a recent heart attack or stroke, varicose veins, or from inactivity or prolonged bed rest. A narrowed or blocked vein in which blood gathers and clots may also cause DVT. This condition can be brought on by an injury to the vein (e.g., such as a sharp blow to the leg), or following surgery or radiation therapy (e.g., chemotherapy). In addition, DVT occurs during some pregnancies due to an increased tendency for the blood to form clots, a natural mechanism to prevent excessive bleeding during childbirth.

Compression sleeves may be used to assist in the treatment of DVT. One example of a compression sleeve suitable for use in treating DVT is disclosed in U.S. Pat. No. 5,025,781, assigned to Microtek Medical Holdings, Inc. Compression sleeves provide gentle, cyclical compression on a body part, such as a calf or thigh, to improve blood circulation within the body part.

There is a tendency in the art to attempt to reuse compression sleeves, recondition used compression sleeves, and resell used, reconditioned compression sleeves. The process of reusing, reconditioning and reselling compression sleeves leads to potential safety problems in the health care industry.

This tendency in the art to attempt to reuse, recondition, and resell used, devices is not limited to compression sleeves. A variety of medical devices are reused, reconditioned, and resold to recycle used devices. As with the reuse of compression sleeves, the process of reusing, reconditioning and reselling devices, such as medical devices, leads to potential safety problems in the health care industry.

There is a need in the art for medical devices, such as compression sleeves, that provides the benefits of conventional medical devices, but enable controlled reuse of the medical device to minimize potential safety problem.

### SUMMARY OF THE INVENTION

The present invention addresses some of the difficulties and problems discussed above by the discovery of controlled reuse medical devices comprising water-soluble material. The medical devices of the present invention comprise one or more water-soluble materials strategically placed within the medical device such that attempts to recycle, reuse, recondition, and/or resell the medical device renders the medical device unusable (i.e., inoperable) for its particular purpose.

In one exemplary embodiment of the present invention, the medical device comprises an inflatable sleeve, such as a compression sleeve, comprising water-soluble material. In this exemplary embodiment, the water-soluble component may comprise water-soluble thread, such as polyvinyl alcohol thread, which dissolves in an aqueous bath having a bath temperature of greater than about 37° C. Attempts to disinfect the inflatable sleeve during a wash cycle in an aqueous bath at a bath temperature of greater than about 37° C. leads to the partial destruction of the inflatable sleeve. The water-soluble thread dissolves and results in a dismantling of the components forming the inflatable sleeve. The resulting components of the inflatable sleeve cannot be used for its particular purpose (i.e., forming an inflatable sleeve to apply pressure of a body part) without reattaching (i.e., resewing) the components of the inflatable sleeve to one another.

In a further exemplary embodiment of the present invention, the medical device may comprise a metal or plastic substrate having a water-soluble coating on an outer surface of the metal or plastic substrate. The water-soluble coating may provide one or more benefits, such as providing a non-stick coating, providing an insulating layer, or providing a fluid-impervious layer. Without the water-soluble coating, the medical device is unacceptable for its particular purpose. When disinfecting the coated medical device after use, the water-soluble coating is removed from the medical device rendering the medical device unsuitable for use.

The present invention is also directed to methods of making and using the above-described medical devices comprising water-soluble material. In one exemplary method of the present invention, the method comprises forming an inflatable sleeve by joining fabric layers to one another using a water-soluble thread, such as polyvinyl alcohol thread. In another exemplary method of the present invention, the method comprises forming a water-soluble coating on at least one surface of a medical device.

The present invention is further directed to a method of rendering a medical device unusable for its particular purpose. In one exemplary method of the present invention, the method comprises exposing the medical device to an aqueous bath under condition such that at least a portion of the medical device becomes soluble. The method results in a medical device that cannot be reused without further processing, such as replacing the water-soluble component.

These and other features and advantages of the present invention will become apparent after a review of the following detailed description of the disclosed embodiments and the appended claims.

### BRIEF DESCRIPTION OF THE FIGURES

FIG. 1 depicts an exemplary inflatable sleeve of the present invention; and

FIG. 2 depicts a plan view of an exemplary inflatable member that may be used to form the inflatable sleeve of FIG. 1.

### DETAILED DESCRIPTION OF THE INVENTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to medical devices comprising water-soluble components, methods of making the medical devices, and methods of using the medical devices. A description of exemplary embodiments of the present invention is given below.

I. Medical Devices Containing at least One Water-Soluble Component

The present invention is directed to medical devices containing at least one water-soluble component. Typically, the medical device further comprises at least one water-insoluble joined to or coated with the at least one water-soluble component. Suitable medical devices include, but are not limited to, compression sleeves, blood pressure cuffs, pulse oximeter probes, splints, surgical gowns, blankets, scrubs, linens intended for isolated patients, infusor bags, tourniquet cuffs, and surgical instruments such as canulae, laparoscopic instruments. In one desired embodiment of the present invention, the medical device comprises an inflatable sleeve, such as a compression sleeve.

The medical devices of the present invention contain at least one water-soluble component. Suitable water-soluble components include, but are not limited to, water-soluble thread or other mechanical fastener, water-soluble coatings, or a combination thereof. As used herein, the term “water-soluble” refers to components having a degree of solubility in water at a water temperature of 37° C. or above. Desirably, the water-soluble component has a degree of solubility in water at a water temperature of 50° C. or above, more desirably, at a water temperature of 75° C. or above, even more desirably, at a water temperature of 90° C. or above.

Water-soluble components used to form at least a portion of the medical devices of the present invention may comprise one or more water-soluble materials. Suitable water-soluble materials for use in the present invention include, but are not limited to, polyvinyl alcohol; polyacrylic acid; polymethacrylic acid; polyacrylamide; water-soluble cellulose derivatives such as methyl celluloses, ethyl celluloses, hydroxymethyl celluloses, hydroxypropyl methyl celluloses, and carboxymethyl celluloses; carboxymethylchitin; polyvinyl pyrrolidone; ester gum; water-soluble derivatives of starch such as hydroxypropyl starch and carboxymethyl starch; and water-soluble polyethylene oxides. Suitable alkali water-soluble materials for use in the present invention include, but are not limited to, ethylene copolymers of acrylic acid (EAA) and methacrylic acid (EMAA), and salts thereof; and ionomers containing acrylic acid and/or methacrylic acid. Desirably, the water-soluble material comprises polyvinyl alcohol with or without acetyl groups, cross-linked or uncross-linked. Suitable polyvinyl alcohol materials are described in U.S. Pat. Nos. 5,181,967; 5,207,837; 5,268,222; 5,620,786; 5,885,907; 5,891,812; the disclosures of all of which are hereby incorporated in their entirety by reference.

In some cases, the water-soluble component used to form at least a portion of the medical devices of the present invention may comprise at least one water-soluble material in combination with at least one water-insoluble material. When the water-soluble component contains both water-soluble and water-insoluble materials, the combined materials are configured so that the mixture is “water-dispersible.” As used herein, the term “water-dispersible” refers to a composite material, which typically contains water-soluble material in combination with water-insoluble material, and is capable of forming a dispersion in an aqueous bath at or above ambient temperature (about 20° C.) and, in some cases, in an aqueous bath at or above ambient temperature (about 20° C.) and having a pH of above 7.0. Desirably, the water-dispersible component is dispersible in water at a water temperature of 37° C. or above, more desirably, at a water temperature of 50° C. or above, even more desirably, at a water temperature of 75° C. or above, and even more desirably, at a water temperature of 90° C. or above.

Suitable water-insoluble materials for use in forming water-dispersible components of the present invention include, but are not limited to, polyurethane resin, ion exchange resins, sodium polyacrylate, polymaleic acid, ammonium polyacrylate, microbial polyesters, polyhydroxybutyrate, polyhydroxybutyrate-valerate, polyhydroxy-alkanoates, polyesters, polyglycolic acid, polyhydroxy acids, aliphatic polyesters, aromatic polyesters, aliphatic-aromatic copolyesters, aliphatic polyetheresters, aromatic polyetheresters, aliphatic-aromatic copolyetheresters, aliphatic polyesteramides, aromatic polyesteramides, aliphatic-aromatic copolyesteramides, aliphatic polyetherester amides, aromatic polyetherester amides, aliphatic-aromatic copolyetherester amides, polyethylene terephthalate, cellulose acetates, polycaprolactone, starch, starch blends, or mixtures thereof, polystyrene, nylon, polyester, polyolefin, polypropylene, polycarbonate, acrylonitrile butadiene styrene, polyethylene, ethylene vinyl acetate copolymer, ethylene methacrylate copolymer, ethylene olefin copolymer, cotton, rayon, cellulose or a mixture.

The water-dispersible component of the medical devices of the present invention may contain any of the above-described water-soluble materials alone or in combination with any of the above-described water-insoluble materials. Desirably, the construction of the water-dispersible component is such that the water-dispersible component either (1) completely dissolves or (2) breaks up into small particles when exposed to conditions, which cause the water-soluble component of the water-dispersible component to become soluble.

In some embodiments of the present invention, the water-dispersible component used to form the medical device comprises water-soluble material alone or in combination with water-insoluble material. When water-insoluble materials are used to form a water-dispersible component, desirably less than about 50 parts by weight (pbw) of water-insoluble material is used in combination with at least about 50 parts by weight (pbw) of water-soluble material to form the water-dispersible component, based on a total parts by weight of the water-dispersible component. More desirably, the water-dispersible component comprises at least about 70 pbw of water-soluble material and less than about 30 pbw of water-insoluble material, even more desirably, at least about 90 pbw of water-soluble material and less than about 10 pbw of water-insoluble material, based on a total parts by weight of the water-dispersible component.

In a further embodiment, the water-dispersible component used to form the medical devices of the present invention consists essentially of water-soluble material. In yet a further embodiment, the water-dispersible component used to form the medical devices of the present invention consists of water-soluble material.

The water-soluble or water-dispersible component may be strategically placed within the medical device such that attempts to recycle, reuse, recondition, and/or resell the medical device renders the medical device unusable (i.e., inoperable) for its particular purpose. For example, the water-soluble or water-dispersible component may be in the form of a mechanical fastener, which connects two or more portions of the medical device to one another. Removal of the mechanical fastener results in unconnected components used to form the medical device. As described in more detail below, suitable mechanical fasteners might include, but are not limited to, thread, adhesives, hook and loop materials, zippers, drawstrings, snaps, and buttons.

In other examples of the present invention, the water-soluble or water-dispersible component may be in the form of a protective coating, which coats a surface of a water-insoluble portion of the medical device. Removal of the coating renders the medical device unsuitable for its intended purpose due to safety and/or contamination concerns to one.

II. Exemplary Medical Devices Containing at least One Water-Soluble Component

The present invention is directed to a variety of medical devices containing at least one water-soluble component. A number of exemplary medical devices are described below.

A. Inflatable Sleeves

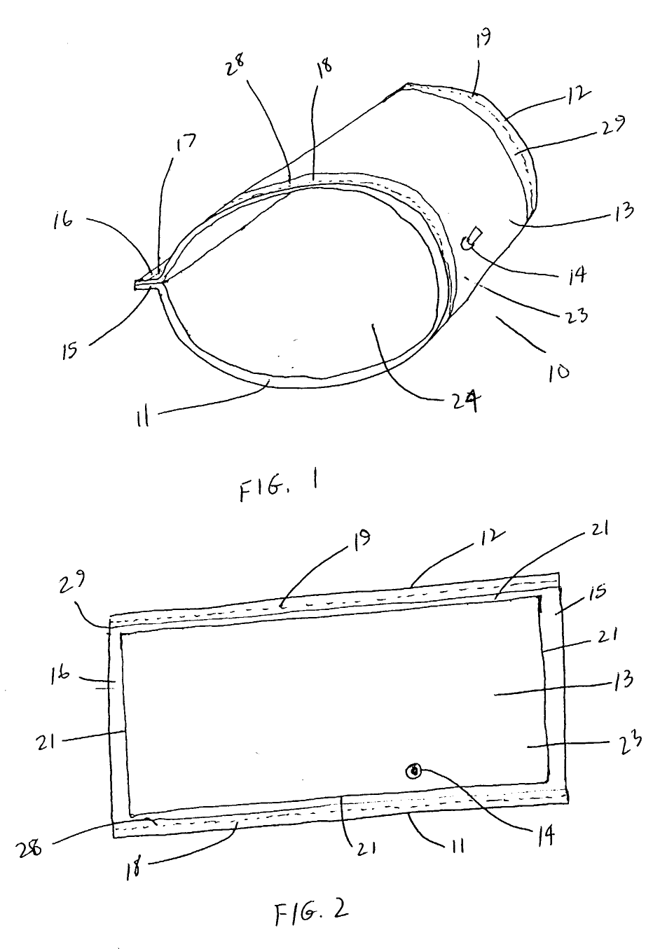

In one desired embodiment of the present invention, the medical device comprises an inflatable sleeve, such as a compression sleeve used in the treatment of deep vein thrombosis (DVT). Suitable inflatable sleeves include, but are not limited to, compression sleeves, blood pressure cuffs, and tourniquet cuffs. An exemplary inflatable sleeve is shown in FIG. 1. As shown in FIG. 1, exemplary inflatable sleeve 10 comprises inflatable member 13 having first end 15 joined to second end 16 to form a tubular-shaped article (i.e., a sleeve). First end 15 may be joined to second end 16 by one or more of the above-mentioned mechanical fasteners. Desirably, first end 15 is joined to second end 16 via water-soluble thread 17. More desirably, water-soluble thread comprises polyvinyl alcohol thread.

Exemplary inflatable sleeve 10 further comprises fluid inlet 14, which may be used to allow flow of fluid into and out of inflatable member 13. Typically, the fluid comprises air; however, other fluids can be used. Suitable fluids include, but are not limited to, water, air, other gases (e.g., CO₂, N₂, etc.), and oils. Exemplary inflatable sleeve 10 also comprises a first conduit end 11 and a second conduit end 12; outer surface 23 and inner sleeve surface 24; optional first seam material 28 and second seam material 29; and optional first conduit end mechanical fastener 18 and second conduit end mechanical fastener 19. When present, mechanical fastener 18 and mechanical fastener 19 are desirably thread, and in some embodiments, water-soluble thread, such as polyvinyl alcohol thread.

In one embodiment of the present invention, exemplary inflatable sleeve 10 is used as a compression sleeve for the treatment of deep vein thrombosis (DVT). In this embodiment, inflatable sleeve 10 is slipped over a body part, such as a calf or thigh (not shown), so that a first portion of a body extends from first conduit end 11 and a second portion of the body extends from second conduit end 12. Air (or other fluid) is forced into inflatable member 13 through fluid inlet 14 resulting in pressure exerted on the body part as inflatable member 13 expands. Inflatable member 13 remains pressurized for a desired period of time, typically less than about 15 seconds. Air (or other fluid) is then allowed to exit inflatable member 13 through fluid inlet 14, resulting in a decrease in pressure exerted on the body part. The gentle, cyclical compression on the body part results in improved blood circulation within the body part.

In the above compression sleeve embodiment, the medical device (i.e., the compression sleeve) provides a particular use or function, namely, cyclical compression on a body part due to the inflation and deflation of inflatable member 13, which surrounds the body part. If a water-soluble component is removed from inflatable sleeve 10, such as water-soluble thread 17, inflatable sleeve 10 can no longer provide its particular use or function described above. In order to be able to again provide its particular use or function, thread or some other mechanical fastener would need to be used to reconnect first end 15 to second end 16 to form an inflatable sleeve 10.

Although inflatable sleeve 10 as shown in FIG. 1 has been described as having only one water-soluble component (i.e., water-soluble thread 17), it should be noted that other components of inflatable sleeve 10 may also be formed of water-soluble material. For example, mechanical fasteners 18 and 19 may be formed from water-soluble material, such as water-soluble thread. Further, inner sleeve surface 24 may be covered with a water-soluble fabric, such as a spunlaced nonwoven fabric formed from polyvinyl alcohol fibers, to provide comfort to a user. However, the only water-soluble component needed to alter to reusability of the above-described inflatable sleeve 10 is water-soluble thread 17. Without water-soluble thread 17, inflatable sleeve 10 transforms into flat, inflatable member 13, which is not capable of surrounding a body part and applying cyclical compression of the body part without some means to surround the body part.

FIG. 2 provides a view of outer surface 23 of inflatable member 13 when water-soluble thread 17 (or some other mechanical fastener) is not present. As shown in FIG. 2, inflatable member 13 comprises first end 15, second end 16, first conduit end 11, second conduit end 12, optional first seam material 28, optional second seam material 29, optional first conduit end mechanical fastener 18, optional second conduit end mechanical fastener 19, and fluid inlet 14. A portion of inflatable member 13 outlined by boundary 21 is inflatable. Typically, two sheets of material are bonded to one another along boundary 21 to form inflatable member 13. The two sheets of material may comprise materials including, but not limited to, sealed fabrics, films, a fabric/film composite, or any other fluid-impermeable material. For example, the two sheets of material used to form inflatable member 13 may comprise first and second sheets of USP vinyl having a thickness of about 0.010 to about 0.012 inches, wherein the first and second sheets of USP vinyl are heat-sealed together (i.e., along boundary 21 as shown in FIG. 2) to provide at least one fluid chamber. Alternatively, the two sheets of material used to form inflatable member 13 may comprise first and second sheets of polyvinyl chloride film having a desired thickness, wherein the first and second sheets of polyvinyl chloride film are heat-sealed together (i.e., along boundary 21 as shown in FIG. 2) to provide at least one fluid chamber.

A first outer fibrous layer may be used to cover at least a portion of the outermost surface of at least one of the two sheets used to form inflatable member 13 to provide an aesthetically pleasing feel and appearance. For example, an outer shell of woven nylon fabric may be used to cover the outer sheet of polyvinyl chloride film. Further, a second outer fibrous layer may be used to cover at least a portion of the outermost surface of the other sheet used to form inflatable member 13 to provide an aesthetically pleasing feel and appearance to inner sleeve surface 24. For example, a flocked layer of fibers may be used to cover the inner sheet of polyvinyl chloride film to form inner sleeve surface 24. Typically, the first outer fibrous layer comprises a woven, knitted or nonwoven fabric layer, such as the above-referenced woven nylon fabric, while the second outer fibrous layer comprises a nonwoven fabric layer or a layer of flocked fibers.

In one desired embodiment of the present invention, the medical device comprises a compression sleeve as described above, wherein at least a portion of the thread used to form the sleeve is polyvinyl alcohol thread. In this embodiment, the sleeve may be sewn along first conduit end 11 and second conduit end 12 (see FIGS. 1-2) using a water-insoluble thread, such as a polyester thread, while first end 15 and second end 16 are connected to one another using a water-soluble thread, such as a polyvinyl alcohol thread. Suitable polyester threads include, but are not limited to, Wildcat Plus® T-35 polyester thread available from Zabin Industries, Inc. (Los Angeles, Calif.). Suitable polyvinyl alcohol threads include, but are not limited to, polyvinyl alcohol threads available from Nitivy Co., Ltd. (Tokyo, JP) under the trade designation SOLVRON®. Suitable SOLVRON® PVA threads for use in the present invention include, but are not limited to, the multifilament threads having the following designations SH, SM, SL, SX, SU, SS and SP, and monofilament threads having the following designations MH and ML. Desirably, the SOLVRON® PVA thread used in the present invention comprises a SL multifilament (100/30/3) polyvinyl alcohol (PVA) thread.

In a further embodiment, the compression sleeve of the present invention is sewn along first conduit end 11 and second conduit end 12 (see FIGS. 1-2) using a Wildcat Plus® T-35 polyester thread, while first end 15 and second end 16 are connected to one another using a SOLVRON® SL multifilament (100/30/3) polyvinyl alcohol (PVA) thread in combination with Wildcat Plus® T-35 polyester thread. In this embodiment, first end 15 and second end 16 are connected to one another using a three-stitch surge sewing machine to form a stitch, wherein the PVA thread is on the needle, and the polyester thread is on the side and bottom loopers. The resulting stitch structure falls apart when exposed to water having a water temperature equal to or greater than about 70° C. (about 158° F.). Desirably, the stitch construction comprises about 14 stitches per inch.

It should be noted that the water-insoluble polyester thread may be replaced with any other commercially available water-insoluble thread. Further, it should be noted that any conventional sewing machine may be used to form the seams (i.e., stitching) in the above-described medical devices.

B. Coated Medical Devices

Other medical devices of the present invention include coated devices, wherein at least a portion of the coating is a water-soluble material as described above. Typically, the water-soluble coating is present to provide a protective coating on at least a portion of the medical device. Removal of the protective coating renders the medical device unsuitable for its intended purpose.

In this embodiment of the present invention, the coated medical device may comprise any of the above-mentioned medical devices having a rigid, coating surface. In other words, the coated medical device is not a coated fabric, a coated film, or a coated fabric/film composite material. In this embodiment, the medical device does not solely comprise fabric and/or film layers, but instead comprises at least one rigid component, such as a metal, plastic, ceramic or glass object having a coatable surface. Desirably, the coated medical device comprises a pulse oximeter probe, a splint, an infusor bag, or a surgical instrument such as a canulae or a laparoscopic instrument.

In one exemplary embodiment of the present invention, the medical device comprises a rigid metal, plastic, glass or ceramic substrate having a water-soluble coating on an outer surface of the rigid metal, plastic, glass or ceramic substrate. The water-soluble coating may provide one or more benefits, such as providing a non-stick coating, providing an insulating layer, or providing a fluid-impervious layer. Without the water-soluble coating, the medical device is unacceptable for its particular purpose. When disinfecting the coated medical device after use, the water-soluble coating is removed from the medical device rendering the medical device unsuitable for its particular use.

C. Medical Devices Containing at least One Mechanical Fastening Device

In a further embodiment of the present invention, the medical device comprises at least one mechanical fastener that is water-soluble or water-dispersible. Desirably, the at least one mechanical fastener is water-soluble. Suitable mechanical fasteners include, but are not limited to, thread, as described above, adhesives, hook and loop materials, zippers, drawstrings, snaps, buttons, or a combination thereof.

In this embodiment of the present invention, the medical device may comprise any of the above-mentioned medical devices. Desirably, the medical device comprises any of the above-described inflatable sleeves, a surgical gown, a blanket, a scrub, a linen, or an infusor bag. Further, in this embodiment of the present invention, the medical device further comprises at least one water-insoluble component that is joined to itself or to another water-insoluble component. For example, the surgical gown, blanket, scrub, or linen may comprise one or more water-insoluble fabric layers joined to other water-insoluble fabric layers or components via at least one mechanical fastener that is water-soluble or water-dispersible.

In one exemplary embodiment of the present invention, the medical device comprises a surgical gown, a blanket, a scrub, a linen, or an infusor bag, wherein two or more water-insoluble portions of the medical device are joined to one another via a water-soluble or water-dispersible mechanical fastener, desirably, a water-soluble mechanical fastener. For example, the medical device may comprise a multi-layer surgical gown or a multi-component surgical gown, wherein water-insoluble layers or water-insoluble components are joined to one another using a water-soluble thread. Upon exposure of the surgical gown to water having a water temperature as described above, the surgical gown breaks up into multiple water-insoluble layers and/or components. The multiple water-insoluble layers and/or components may be reprocessed to form a reusable surgical gown, but further processing (i.e., sewing) is required to reuse the surgical gown.

In a further exemplary embodiment of the present invention, the medical device comprises a blood pressure cuff, wherein water-insoluble portions of the blood pressure cuff are temporarily attached to one another (i.e., to form a cuff around a body part) via at least one water-soluble or water-dispersible mechanical fastener. The at least one water-soluble or water-dispersible mechanical fastener may comprise (i) water-soluble or water-dispersible hook material, (ii) water-soluble or water-dispersible loop material, (iii) water-soluble or water-dispersible adhesive for bonding hook and/or loop material to the blood pressure cuff, (iv) water-soluble or water-dispersible thread for attaching hook and/or loop material to the blood pressure cuff, (v) any other water-soluble or water-dispersible mechanical fastener for attaching hook and/or loop material to the blood pressure cuff, or (vi) a combination thereof. In this embodiment, exposure of the blood pressure cuff to water having a water temperature as described above causes the water-soluble or water-dispersible material to dissolve, rendering the blood pressure cuff unusable for its particular purpose.

II. Methods of Making Medical Devices

The present invention is also directed to methods of making the above-described medical devices comprising at least one water-soluble component. The methods of making medical devices of the present invention comprise strategically incorporating at least one water-soluble or water-dispersible component into a medical device such that removal of the at least one water-soluble or water-dispersible component results in an unusable or inoperable medical device. As used herein, the terms “unusable” and “inoperable” are used interchangeably to describe a medical device that cannot be used for its intended purpose without further processing.

A. Methods of Making Inflatable Sleeves

In one exemplary method of the present invention, the method comprises forming an inflatable sleeve as described above. In this embodiment, the method of making a medical device (i.e., an inflatable sleeve) comprises strategically incorporating at least one water-soluble or water-dispersible mechanical fastener into the medical device (i.e., an inflatable sleeve) to form a tubular article. Desirably, the method comprises joining ends of an inflatable member to one another using a water-soluble thread, such as polyvinyl alcohol thread. As discussed above, the inflatable member may be formed from two sheet materials, wherein each sheet material is a single or multi-layer material that is fluid-impervious (i.e., allows a compartment or fluid chamber formed by the two sheet materials to be inflatable).

The method for forming an inflatable sleeve may further comprise one or more of the following steps:

forming two identical or different sheets of material, wherein each of the sheets of material are fluid-impermeable;

bonding the two sheets of material to one another to form an inflatable member having an inflatable compartment (or fluid chamber) between the two sheets, wherein the inflatable compartment has an outer boundary;

forming an opening in at least one of the two sheets of material within the outer boundary of the inflatable compartment;

attaching a fluid inlet to at least one of the two sheets of material so that the fluid inlet fits in the opening within the outer boundary of the inflatable compartment;

providing a fluid source and tubing for connecting the fluid source to the fluid inlet of the inlet to at least one of the two sheets of material so that the fluid inlet of the inflatable compartment;

attaching one or more devices to the inflatable compartment such as a fluid pressure indicator for measuring the pressure within the inflatable compartment;

optionally, attaching seam material to one or more edges of the two sheets of material, wherein the seam material is outside the outer boundary of the inflatable compartment;

optionally, attaching a fabric or fiber layer onto outer surfaces of the two sheets of material used to form the inflatable compartment;

optionally, contacting an outer surface of at least one of the two sheets of material (or cover fabric or fiber layers thereon) with a printable material (e.g., an ink, dye or pigment); and

testing the inflatability of the inflatable member by inputting a fluid, such as air or water, into the inflatable compartment.

B. Methods of Making Coated Medical Devices

In another exemplary method of the present invention, the method comprises forming a water-soluble or water-dispersible coating on at least one surface of a medical device. The water-soluble or water-dispersible coating may be applied to the medical device via any conventional coating method. Suitable coating methods include, but are not limited to, solution coating, spray coating, dip coating (i.e., dipping the medical device into a coating solution), printing, laminating, extrusion coating, etc.

The method for forming a coated medical device may further comprise one or more of the following steps:

forming a three-dimensional object from metal, plastic, glass and/or ceramic material, wherein the three-dimensional object is not solely a fabric, film or fabric/film composite material;

coating at least a portion of the three-dimensional object with a water-soluble or water-dispersible coating material;

drying the coating material;

optionally, contacting an outer surface of the medical device with a printable material (e.g., an ink, dye or pigment); and

testing the sufficiency (i.e., thickness and/or uniformity) of the coating via one or more test methods.

C. Methods of Making Medical Devices Containing at least One Mechanical Fastening Device

In a further exemplary method of the present invention, the method comprises attaching one or more components of a medical device with at least one water-soluble or water-dispersible mechanical fastener. As described above, the one or more components may be water-insoluble layers or components of the medical device.

The method for forming a medical device containing at least one water-soluble or water-dispersible mechanical fastener may comprise one or more of the following steps:

connecting two or more water-insoluble layers, water-insoluble components, and/or portions of a water-insoluble component of the medical device to one another using at least one water-soluble or water-dispersible mechanical fastener;

attaching one or more components to the medical device such as a label, a fluid source (for inflatable devices), a fluid pressure indicator for measuring the pressure within an inflatable compartment, tubing, etc.;

optionally, contacting an outer surface the medical device with a printable material (e.g., an ink, dye or pigment); and

testing the integrity of the joined water-insoluble components using one or more test methods.

III. Methods of Rendering Medical Devices Unusable

The present invention is further directed to a method of rendering a medical device unusable for its particular purpose. The method of rendering a medical device unusable comprises removing at least one strategically placed water-soluble or water-dispersible component from the medical device. The resulting medical device is not suitable for its intended use as discussed above.

In one embodiment of the present invention, the method of rendering a medical device unusable comprises exposing the medical device to an aqueous bath under condition such that at least one strategically placed water-soluble or water-dispersible component becomes soluble or dispersible. The method results in a medical device that cannot be reused for its particular purpose without further processing, such as replacing the water-soluble or water-dispersible component.

A. Method of Rendering an Inflatable Sleeve Unusable

In one desired embodiment, the medical device comprises an inflatable sleeve, such as a compression sleeve, comprising water-soluble thread, more desirably, polyvinyl alcohol thread. The method of rendering the inflatable sleeve unusable or inoperable comprises exposing the inflatable sleeve to an aqueous bath under condition such that the water-soluble thread becomes soluble. As discussed above, exposing the water-soluble thread to an aqueous bath having a bath temperature of at least about 37° C. (or at least about 50° C., or at least about 75° C., or at least about 90° C.) solubilizes the water-soluble thread. In this embodiment, the method results in an inflatable member that cannot be reused for its particular purpose, namely, as an inflatable sleeve supplying cyclical compression of a body part. The resulting inflatable member can only be used as an inflatable sleeve by replacing the removed thread.

It should be noted that the present invention also encompasses a method of restoring the unusable medical device to a usable state. For example, in the case of an inflatable sleeve, one method of the present invention further comprises rejoining ends (i.e., first end 15 to second end 16 as shown in FIGS. 1-2) of the resulting inflatable member to one another to form an inflatable sleeve. In this method, the ends of the resulting inflatable member may be joined to one another using a water-soluble thread, such as the above-described polyvinyl alcohol thread, a water-insoluble thread, or a combination thereof. The method may further comprise replacing any other water-soluble or water-dispersible component that is removed from the inflatable sleeve during the above-described step of rendering the inflatable sleeve unusable.

The step of rendering the inflatable sleeve unusable may be performed using commercially available washing machines. Suitable washing machines include, but are not limited to, any commercially available washing machine for home, commercial or industrial use. Examples of suitable washing machines include, but are not limited to, washing machines available from Sears, Roebuck and Company (Chicago, Ill.), and washing machines available from Pellerin Milnor Corporation (Kenner, La.). Desirably, the washing machine has a load capacity (i.e., weight of medical devices, not medical devices with water) of at least about 45 kilograms (kg) (100 lbs.), more desirably, at least about 113 kilograms (kg) (250 lbs.), even more desirably, at least about 227 kilograms (kg) (500 lbs.).

The step of rendering the inflatable sleeve unusable may use an aqueous bath having a bath temperature as described above. The aqueous bath may comprise water alone or in combination with one or more additional components. In addition to water, the aqueous bath may include one or more additional components including, but not limited to, surfactants, detergents or other cleaning agents. Commercially available detergents may be used in the washing step. An example of a suitable surfactant is E-500 commercially available from Paragon Corporation (Birmingham, Ala.). An example of a suitable detergent is ASSERT brand detergent, also commercially available from Paragon Corporation (Birmingham, Ala.).

The method of rendering the inflatable sleeve (or any other medical device of the present invention) unusable or inoperable may further comprise one or more of the following steps:

placing the inflatable sleeve into a washer;

introducing water into the washer to form an aqueous solution;

introducing one or more additional components into the aqueous solution;

heating the aqueous solution to a desired temperature;

maintaining the desired bath temperature for a desired period of time, typically, from about 3 minutes to about 8 hours, more typically, from about 3 minutes to about 60 minutes, and even more typically, from about 3 minutes to about 10 minutes; and

(10) removing any insoluble components from the washer.

B. Method of Rendering a Coated Medical Device Unusable

In a further embodiment of the present invention, the method of rendering a medical device unusable comprises exposing a coated medical device to an aqueous bath under condition such that at least one strategically placed water-soluble or water-dispersible coating becomes soluble or dispersible. The method results in a substantially uncoated medical device that cannot be reused for its particular purpose without further processing, such as replacing the coating, whether the replacement coating is water-soluble, water-dispersible, or water-insoluble.

The method of rendering the coated medical device unusable or inoperable comprises exposing the coated medical device to an aqueous bath under condition such that the water-soluble or water-dispersible coating becomes soluble or dispersible. As discussed above, exposing the water-soluble or water-dispersible coating to an aqueous bath having a bath temperature of at least about 37° C. (or at least about 50° C., or at least about 75° C., or at least about 90° C.) solubilizes the water-soluble coating or disperses the water-water-dispersible coating. In this embodiment, the method results in an uncoated medical device that cannot be reused for its particular purpose. The resulting uncoated medical device can only be used as a coated medical device by replacing the removed coating with a new coating.

The present invention also encompasses a method of restoring the unusable article (i.e., the uncoated medical device) to a usable state (i.e., to a coated medical device). For example, in the case of a coated medical device, one method of the present invention further comprises coating the uncoated medical device to form a coated medical device. In this method, the resulting uncoated medical device may be coated using a water-soluble coating, such as a polyvinyl alcohol coating, a water-dispersible, or a water-insoluble coating. The method may further comprise replacing any other water-soluble or water-dispersible component that is removed from the coated medical device during the above-described step of rendering the coated medical device unusable.

C. Method of Rendering a Medical Devices Containing at least One Mechanical Fastening Device Unusable

In a further embodiment of the present invention, the method of rendering a medical device unusable comprises exposing a medical device to an aqueous bath under condition such that at least one strategically placed water-soluble or water-dispersible mechanical fastener becomes soluble or dispersible. The method results in one or more unattached components of a medical device that cannot be reused for its particular purpose without further processing, such as replacing the mechanical fastener(s), whether the replacement mechanical fastener(s) is a water-soluble, water-dispersible, or water-insoluble mechanical fastener(s).

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A medical device containing at least one strategically placed water-soluble or water-dispersible component and having an intended purpose, wherein removal of the at least one strategically placed water-soluble or water-dispersible component renders the medical device unusable for the intended purpose.

2. The medical device of claim 1, wherein the at least one strategically placed water-soluble or water-dispersible component comprises a water-soluble or water-dispersible coating, mechanical fastener, or combination thereof.

3. The medical device of claim 1, wherein the medical device comprises a compression sleeve, a blood pressure cuff, a pulse oximeter probe, a splint, a surgical gown, a blanket, a scrub, a linen, an infusor bag, a tourniquet cuff, a surgical instrument, a canulae, or a laparoscopic instrument.

4. The medical device of claim 3, wherein the medical device comprises one or more water-insoluble components.

5. The medical device of claim 4, wherein the one or more water-insoluble components are connected to one another by the at least one strategically placed water-soluble or water-dispersible component.

6. The medical device of claim 5, wherein the at least one strategically placed water-soluble or water-dispersible component comprises thread, adhesives, hook material, loop material, a zipper, a drawstring, a snap, a button, or a combination thereof.

7. The medical device of claim 4, wherein the one or more water-insoluble components are coated with the at least one strategically placed water-soluble or water-dispersible component.

8. The medical device of claim 1, wherein the medical device comprises an inflatable sleeve and the at least one strategically placed water-soluble or water-dispersible component comprises water-soluble thread used to form a tubular article.

9. The medical device of claim 8, wherein the water-soluble material comprises polyvinyl alcohol thread.

10. The medical device of claim 9, wherein the polyvinyl alcohol thread is soluble in an aqueous bath having a bath temperature of greater than about 37° C.

11. The medical device of claim 9, wherein the polyvinyl alcohol thread is soluble in an aqueous bath having a bath temperature of greater than about 90° C.

12. A deep vein thrombosis compression sleeve comprising the medical device of claim 8.

13. The deep vein thrombosis compression sleeve of claim 12, wherein the compression sleeve further comprises an inner lining covering at least a portion of an inner surface of the inflatable sleeve.

14. The deep vein thrombosis compression sleeve of claim 12, wherein the compression sleeve further comprises at least one fluid inlet.

15. The medical device of Claim l, wherein the at least one strategically placed water-soluble or water-dispersible component comprises a water-soluble coating.

16. The medical device of claim 15, wherein the water-soluble coating comprises polyvinyl alcohol.

17. The medical device of claim 1, wherein the at least one strategically placed water-soluble or water-dispersible component comprises at least one mechanical fastener.

18. The medical device of claim 17, wherein the at least one mechanical fastener comprises polyvinyl alcohol.

19. A deep vein thrombosis compression sleeve comprising water-soluble material.

20. The compression sleeve of claim 19, wherein the water-soluble material comprises polyvinyl alcohol thread.

21. The compression sleeve of claim 20, wherein the polyvinyl alcohol thread is soluble in an aqueous bath having a bath temperature of greater than about 37° C.

22. The compression sleeve of claim 20, wherein the polyvinyl alcohol thread is soluble in an aqueous bath having a bath temperature of greater than about 90° C.

23. A method of rendering a medical device unusable, wherein the method comprises:
exposing the medical device to an aqueous bath under conditions such that at least one strategically placed water-soluble or water-dispersible component of the medical device becomes soluble or disperses.

24. The method of claim 23, wherein the aqueous bath has a bath temperature of greater than about 37° C.

25. The method of claim 23, wherein the aqueous bath has a bath temperature of greater than, about 90° C.

26. The method of claim 23, wherein the medical device comprises a compression sleeve, a blood pressure cuff, a pulse oximeter probe, a splint, a surgical gown, a blanket, a scrub, a linen, an infusor bag, a tourniquet cuff, a surgical instrument, a canulae, or a laparoscopic instrument.

27. The method of claim 26, wherein the medical device comprises one or more water-insoluble components.

28. The method of claim 27, wherein the one or more water-insoluble components are connected to one another by the at least one strategically placed water-soluble or water-dispersible component.

29. The method of claim 28, wherein the at least one strategically placed water-soluble or water-dispersible component comprises thread, adhesives, hook material, loop material, a zipper, a drawstring, a snap, a button, or a combination thereof.

30. The method of claim 27, wherein the one or more water-insoluble components are coated with the at least one strategically placed water-soluble or water-dispersible component.

31. The method of claim 23, further comprising replacing the at least one strategically placed water-soluble or water-dispersible component with a replacement component after the exposing step, wherein the replacement component comprises a water-soluble, water-dispersible, or water-insoluble component.

32. The method of claim 31, wherein the replacement component comprises a water-soluble or water-dispersible component.
